(19) United States
(12) Patent Application Publication
(10) Pub. No.: US 2022/0213432 A1
(43) Pub. Date: Jul. 7, 2022

(54) Transplantable cell composition comprising eukaryotic cells in a nanofibrillar cellulose hydrogel, method for preparing thereof and use of nanofibrillar cellulose

(71) Applicant: UPM-Kymmene Corporation, Helsinki (FI)

(72) Inventors: Markus NUOPPONEN, Helsinki (FI); Tony KIURU, Helsinki (FI)

(21) Appl. No.: 17/611,669

(22) PCT Filed: Jun. 24, 2019
(86) PCT No.: PCT/FI2019/050488

§ 371 Date: Nov. 16, 2021

(51) Int. Cl. C12N 5/00; C12N 5/0793; A61K 35/30

(52) U.S. Cl. CPC C12N 5/0062; C12N 5/062; A61K 35/30; C12N 2506/02; C12N 2533/78; C12N 2539/00; C12N 2513/00

(57) ABSTRACT

The present application provides a method for preparing transplantable cell preparation, the method comprising culturing eukaryotic cells at conditions allowing the cells to coalesce and form cell aggregates, providing the cell aggregates in a nanofibrillar cellulose hydrogel to obtain a transplantable cell composition comprising eukaryotic cells in a nanofibrillar cellulose hydrogel matrix, and a transplantable cell preparation. The present application also provides the transplantable cell composition for use in a therapeutic method, and to use of nanofibrillar cellulose for preparing the transplantable cell composition.

### FIELD OF THE APPLICATION

The present application relates to a transplantable cell composition comprising eukaryotic cells in a nanofibrillar cellulose hydrogel matrix, and to a method for preparing thereof. The present application also relates to the transplantable cell composition for use in a therapeutic method, and to use of nanofibrillar cellulose for preparing the transplantable cell composition.

### BACKGROUND

Transplantation of stem cells may be required in medical treatment, for example cancer treatment or other treatment requiring regeneration of certain cells or tissue. However, it is challenging to obtain a suitable environment and composition for the transplantable cells, which could retain transplanted stem cells in place and localize cell-to-cell signalling. In a tissue extracellular matrix (ECM) is secreted by cells and surrounds them in tissues. It is structural support for cells having characteristics features of the tissue. It is not simply a passive, mechanical support for cells, but complex scaffold including a variety of biologically active molecules that are highly regulated and critical for determining the action and fate of the cells that it surrounds. However such matrix is not useful for most transplantation uses because of the variety of contained biological molecules and structures, which may cause rejection or other undesired reactions in the recipient's body.

In some cases matrices such as mammal-derived collagens have been used, as they provide local environment cues present in mammalian tissue. However, they are unstable, for example vulnerable to in vivo enzymatic degradation, making it difficult to create long-lasting niche. Therefore it is desired to find durable and compatible transplantable compositions and methods for providing cells to a subject.

### SUMMARY

In the present application it is disclosed how nanofibrillar cellulose hydrogel can be used with stem cells, such as stem-cell derived spheroids, in cell transplantation to reduce undesired agglomeration and insulate cells from injection shearing forces that may cause apoptosis. Nanofibrillar cellulose hydrogel derived matrix materials show great potential for in vivo applications.

The present application provides a method for preparing transplantable cell preparation, the method comprising
culturing eukaryotic cells at conditions allowing the cells to form cell aggregates, preferably for at least three days,
providing the cell aggregates in a nanofibrillar cellulose hydrogel and/or combining the cell aggregates with a nanofibrillar cellulose hydrogel to obtain a transplantable cell composition comprising eukaryotic cells in a nanofibrillar cellulose hydrogel matrix.

The present application provides a transplantable composition comprising a nanofibrillar cellulose hydrogel matrix.

The present application provides a transplantable cell composition comprising eukaryotic cells in a nanofibrillar cellulose hydrogel matrix. This cell composition may be obtained with the method for preparing transplantable cell preparation.

The present application provides the transplantable cell composition for use in a therapeutic method comprising administering cells to a subject.

The present application provides use of nanofibrillar cellulose for preparing the transplantable cell composition.

The main embodiments are characterized in the independent claims. Various embodiments are disclosed in the dependent claims. The embodiments and examples recited in the claims and in the specification are mutually freely combinable unless otherwise explicitly stated.

The nanofibrillar cellulose, which is present as a hydrogel, forms a hydrophilic interstitial matrix for cells, which matrix is non-toxic, biocompatible and also biodegradable. The matrix can be degraded enzymatically, for example by adding cellulase. On the other hand the hydrogel is stable at physiological conditions, and does not need to be crosslinked by using additional agents. The properties, such as permeability, of the nanofibrillar cellulose hydrogel may be controlled by adjusting the chemical and/or physical properties of the nanofibrillar cellulose.

Certain advantageous properties of the hydrogel comprising nanofibrillar cellulose include flexibility, elasticity and remouldability, which enable for example injecting the hydrogel into a variety of targets in a body. As the hydrogel contains a lot of water, it also shows good permeability for molecules. The hydrogels of the embodiments also provide high water retention capacity and molecule diffusion property speed.

The nanofibrillar cellulose hydrogels described herein are useful in medical and scientific applications, wherein the materials comprising nanofibrillar cellulose are in contact with living matter. The products containing nanofibrillar cellulose as described herein are highly biocompatible with the living matter and provide several advantageous effects. Without binding to any specific theory, it is believed that a hydrogel comprising very hydrophilic nanofibrillar cellulose having a very high specific surface area, and thus high water retention ability, when applied against cells, provides favourable moist environment between the cells and the hydrogel comprising nanofibrillar cellulose. The high amount of free hydroxyl groups in the nanofibrillar cellulose forms hydrogen bonds between the nanofibrillar cellulose and water molecules and enables gel formation and the high water retention ability of the nanofibrillar cellulose. The nanofibrillar cellulose hydrogel contains a high amount of water, and it also enables migration of fluids and/or agents.

The nanofibrillar cellulose may be used as a matrix for cells thus providing an environment, which protects the cells and helps them to maintain their viability. The formed matrix, which may be called interstitial matrix, resembles ECM and provides a meshwork-like matrix with heterogenous pore sizes. The dimensions of the network of cellulose nanofibrils is very close to natural ECM network of collagen nanofibrils. It provides structural support for cells and a network of interconnected pores for efficient cell migration and transfer of nutrients to the cells. Furthermore, nanofibrillar cellulose is non-animal-based material and therefore xeno-free, so there is no risk for disease transfer or rejection. Especially when human cells are concerned, the formulation will comprise, in addition to the cellulose and probably minor amount of additives, only human-derived components, so it does not contain any material from foreign animal or microbial species.

Cellulose nanofibrils have negligible fluorescence background. With the present materials it is possible to obtain a transparent and porous matrix for the cells, and the handling of the material is easy compared to the alternatives. Cellulose nanofibril hydrogel has optimal elasticity, stiffness, shear stress, mechanical adhesion and porosity to be also used as 3D and 2D cell storage or culture matrix.

Nanofibrillar cellulose hydrogels are injectable and transplantable and thus capable of delivering cells, such as stem cells to a desired subject. Nanofibrillar cellulose hydrogels are pseudoplastic materials which makes them easily injectable, as the extruding shearing force is large enough to reduce viscosity during injection, and after the injection, when the shearing force is removed, the material will stabilize to retain its shape. When injected or implanted, the cells remain in the subject in an active form. The use of nanofibrillar cellulose as interstitial matrix prevents or decreases undesired agglomeration of the cells or cell spheroids.

Cellulose is biocompatible due to moderate, if any, foreign responses and is safe for stem-cell applications, with no known toxicity. It is also biodurable; cellulose resorption is slow, as cells cannot synthesize cellulases required to degrade cellulose, and thus nanofibrillar cellulose hydrogels will remain localized.

### BRIEF DESCRIPTION OF THE FIGURES

FIG. 1 shows an exemplary confocal photomicrograph of a hESC-derived ONP spheroid generated with GrowDex™ is shown. Scale bar=250 μm.

FIG. 2 shows an exemplary confocal photomicrograph of a hESC-derived ONP spheroid generated with GrowDex-T™ is shown. Scale bar=250 μm.

FIG. 3 shows (A): Human PSC-derived ONPs can be generated in a 3-D culture environment to form a spheroid with 2% GrowDex-T™ (likely increasing survival in ex-vivo and in vivo). βIIIT (βIII-tubulin) and MAP2 are human ONP markers. Scale bar=100 μm. (B): Human PSC-derived AN progenitors can be neuronally differentiated in a hydrogel-created stem cell niche. White arrows indicate neuronal connectivity. Scale bar=20 μm. (C): Human PSC-derived ANs can be identified with RFP (expressed in CP: cytoplasm) and a nucleus (N: nucleus). PAX2: counter-stained. Scale bar=20 μm. (D): Human hESC-derived ONP spheroids transplanted with 1.5% GrowDex™ can be also identified with STEM101 (ST101), anti-human nuclear antibody in the inner ear (mouse). Note that STEM101 positive cells are found in the scala media (SM), one of the three chambers in the inner ear, after a transplantation surgery with hPSC-derived ONPs. TOTO3 (TOTO3 iodide): counter-stained.

### DETAILED DESCRIPTION

In this specification, percentage values, unless specifically indicated otherwise, are based on weight (w/w). If any numerical ranges are provided, the ranges include also the upper and lower values. The open term “comprise” also includes a closed term “consisting of” as one option.

The materials and products described herein may be medical and/or scientific materials and products, such as life science materials and products, and may be used in the methods and the applications involving living cells and/or bioactive material or substances, such as described herein. The materials or products may be or relate to cell transplantation, cell culture, cell storage and/or cell study materials or products, and may be used in methods wherein cells are transplanted, cultured, stored, maintained, transported, provided, modified, tested, and/or used for medical or scientific purposes, or in other related and applicable methods.

The present application presents a composition comprising cells in a nanofibrillar cellulose hydrogel. The composition is transplantable, i.e. it is in a form which enables safe delivering or administering the cells to a subject to provide or form cell transplant. It is not desired that a cell transplant would cause rejection or disease in the subject. The present application also provides a transplantable composition comprising a nanofibrillar cellulose hydrogel matrix. Such a composition may or may not contain cells, and it may be provided as an intermediate product or raw material, and it may be packed in a suitable sealed package for storing, transport and/or use.

A transplantable composition preferably has a specific chemical content, dry content and type of the matrix material. A transplantable composition, or the nanofibrillar cellulose hydrogel in the composition, preferably does not include other type of cell-derived material, such as cell culture medium or material derived from cell culture medium, or it may only include traces of such material. The cell-derived material may include cells, cell organelles, hormones, antigens, proteins, peptides, nucleic acids and/or lipids of origin other than the transplantable cells.

Preferably the composition or the nanofibrillar cellulose hydrogel contains no or only traces of other type of cell-derived material. A “trace” may refer to less than 0.5% (w/w), less than 0.1% (w/w), less than 0.05% (w/w) or less than 0.01% (w/w), or to amounts which cannot be detected using common methods for detecting or identifying specific biological compounds, such as immunological methods. The composition, and/or the nanofibrillar cellulose hydrogel in the composition, is preferably animal origin free, xeno-free and/or feeder-free. Feeder-free refers to cell culture medium and/or conditions wherein feeder cells are not present, for example wherein cells are cultured in absence of feeder cell layer.

The composition may be an injectable composition or an implantable composition. The composition may be provided in a suitable form, such as in a syringe, micropipette or other suitable applicator. One embodiment provides the transplantable cell composition packed in a syringe or micropipette. The syringe may be any suitable syringe, which may also include an injection needle. In one embodiment the syringe comprises an injection needle for transplanting the composition into a subject. The syringe and the needle may be used in the therapeutic methods discussed herein, such as in the cell transplantation, and also for storing, transporting and providing the transplantable cell composition. The present application also provides a syringe comprising the transplantable cell composition, preferably comprising an injection needle for transplanting the composition into a subject. The micropipette may be any micropipette suitable for cell transplantation, such as cell transfer micropipette. The applicators disclosed herein may be operated or operable by using pressure-based or volume-based instruments, such as an injector or a pump. The composition may be provided as a dose, such as packed in an applicator such as syringe or micropipette, or micropipette tip. The dose may have a volume of 500 μl or less, such as 10-500 μl, 10-200 μl or 20-200 μl.

The composition may be also packed in a tube, which may be any suitable tube, such as a sealable test tube, which may be used for storing, transporting and providing the transplantable cell composition. One example provides an implant comprising cells in a nanofibrillar cellulose hydrogel, preferably comprising the transplantable cell composition described herein. More particularly the transplantable cell composition may be included in an implant comprising one or more reinforcing part(s). The nanofibrillar cellulose may be in the implant in the concentrations disclosed for injectable compositions, such as 1-3% (w/w), or the implant may contain a higher concentration of nanofibrillar cellulose, such as 10% or less, such as 1-10% (w/w), 4-10% (w/w), 5-10% (w/w), or 3.5-8% (w/w).This would help the implant to maintain its form and facilitate implanting.

Transplantation as used herein refers to transfer (engraftment) of cells from a donor to a recipient with the aim of restoring function(s) in the body. When transplantation is performed between different species, e.g. animal to human, it is named xenotransplantation. The donor may be the same or different as the recipient. The transplantation may be autologous or allogenic.

The cells, especially eukaryotic cells, may be stem cells or differentiated cells, such as cells originated or derived from human or animal body, for example from a patient. The cells may be autologous cells, in which case the cells are obtained from the same subject that will receive the cells in the therapy. This may be implemented for example in autologous stem cell transplantation, also called autogenous, autogeneic or autogenic stem cell transplantation, which is transplantation of stem cells, wherein the stem cells are removed from a subject, stored, and later given back to that same subject. Therefore the donor and the recipient of the cells is the same subject or individual, such as a person. Autologous cells may have been treated and/or modified after removing from the subject and before returning back to the subject. In one example in autologous stem cell transplantation the subject's own cells are removed or harvested before a treatment that destroys them from the body, such as cancer treatment by chemotherapy and/or radiation, the harvested cells are stored, for example frozen and subsequently thawn, and finally returned back to the body. This kind of transplant is mainly used during treatment of certain leukemias, lymphomas, and multiple myeloma, and sometimes also in treatment of other cancers, like testicular cancer and neuroblastoma, and certain cancers in children.

Alternatively the cells may be allogenic cells, in which case the cells are obtained from different subject (donor) from the subject (recipient) that will receive the cells in the transplantation or other therapy.

The stem cells may be singe or separate stem cells, which are not aggregated, or stem cell spheroids, such as stem cell derived spheroids. In one embodiment the stem cells are human stem cells.

Cell spheroids refer to multicellular cell aggregates linked together by extracellular matrix, in this case multicellular cell aggregates linked together by nanofibrillar cellulose matrix. Spheroids are more complex than single cells present as separate cells due to dynamic cell-cell and cell-matrix interaction which makes them an important tool for resembling the in vivo tissues microenvironment in vitro. Cell spheroids can be formed by culturing or incubating cells in a matrix material to form three-dimensional cell culture system(s) containing multicellular aggregates or spheroids. The matrix material may be a suitable gel material, preferably hydrogel, such as agarose gel or nanofibrillar cellulose gel. When nanofibrillar cellulose was used as the matrix material cell spheroids could be obtained already after three days of culturing or incubating. In general the diameter of cell spheroids may vary and range from tens of micrometres to over millimetre. However herein cell spheroids with a controlled diameter suitable for transplantation purposes were obtained by controlling and adjusting the culturing and matrix formation materials and conditions.

Cell spheroids are preferred as it was found out that in the transplantation process the multicell aggregates stabilized by the fibrillar cellulose network facilitated the activity, proliferation and differentiation of the cells in the subject. The nanofibrillar cellulose matrix created optimal conditions for transplantation purposes for such cell aggregates. The cell aggregates formed in the methods disclosed herein may be in the form of cell spheroids, or the cell aggregates may form cell spheroids.

The present application provides a method for preparing transplantable cell preparation, the method comprising
culturing eukaryotic cells at conditions allowing the cells to form cell aggregates,
providing the cell aggregates in a nanofibrillar cellulose hydrogel to obtain a transplantable cell composition comprising eukaryotic cells in a nanofibrillar cellulose hydrogel.

In one embodiment, the method comprises
culturing eukaryotic cells at conditions allowing the cells and form cell aggregates,
combining the cell aggregates with a nanofibrillar cellulose hydrogel to obtain a transplantable cell composition comprising eukaryotic cells in a nanofibrillar cellulose hydrogel.

The method may comprise culturing the cells at conditions allowing the cells to coalesce. The conditions include suitable time to allow the coalescing and/or the formation of the aggregates. The conditions may include a suitable culturing medium, such as liquid medium and/or gel medium. The culturing may be carried out in a culture dish or plate, or in a multiwell microplate. The cells may be cultured in hydrogel, such as nanofibrillar cellulose hydrogel, which preferably contains liquid culturing medium. Using NFC hydrogel may facilitate obtaining cell spheroids of desired size. The concentration of the hydrogel shall not bee too high as too high concentration of the gel matrix material, such as NFC, may prevent spheroid formation, especially with stem cells, such as pluripotent stem cells. Preferably the concentration of the hydrogel is not more than 3% (w/w), or not more than 2.5% (w/w). In examples the cells are cultured in about 1% (w/w) NFC hydrogel, in about 1.5% (w/w) NFC hydrogel or in about 2% (w/w) NFC hydrogel. The method may comprise culturing the eukaryotic cells in nanofibrillar cellulose hydrogel having a concentration in the range of 0.8-3% (w/w), 1.3-2.2% (w/w), 0.8-2%, 1-2% (w/w), such as 0.8-1.5% (w/w), or about 1% (w/w), about 1.5% (w/w) or about 2% (w/w). The cells may be seeded or provided to the cell culture in a density of 1×10⁵ cells/ml-1×10⁷ cells/ml, such as in a density of 1×10⁶-5×10⁶ cells/ml.

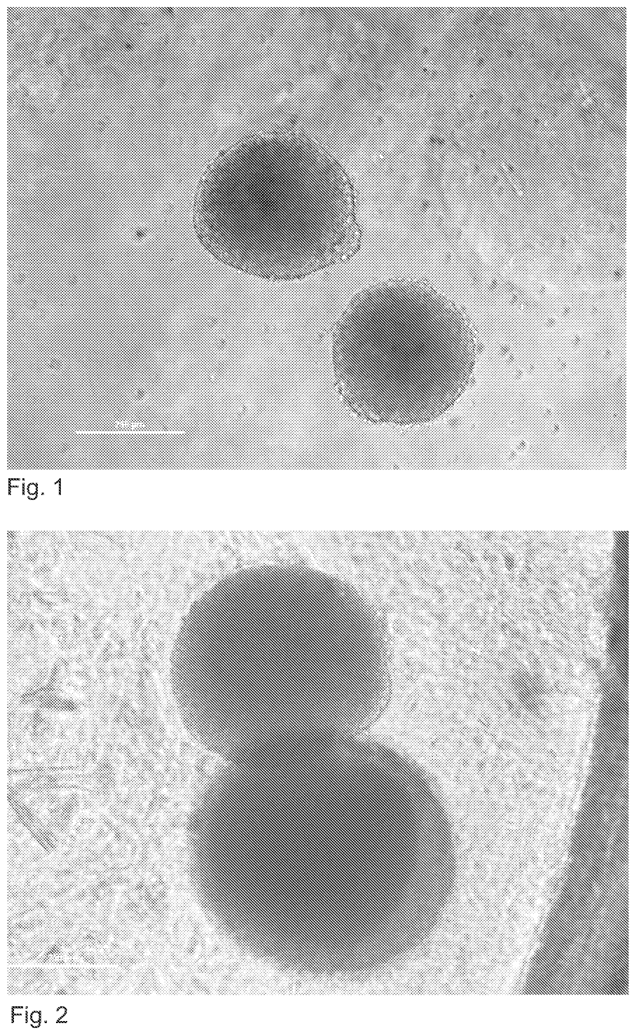

The cell aggregates, which may be cell spheroids, may have an average diameter in the range of 80-700 μm, such as in the range of 80-300 μm as shown in FIGS. 1, 2 and 3A. It seems that cell spheroids generated with NFC, such as cultured in the NFC and/or combined with the NFC, having a concentration of about 2% (w/w) had a smaller average diameter, such as in the range of 80-250 μm, than cell spheroids generated in NFC with a lower concentration. The diameter could also be maintained. This may increase the survival of the cells ex-vivo and in-vivo and enables providing efficient and viable transplantable cell preparations. It is also possible to adjust the diameter of the formed or forming cell spheroids, for example to reduce the diameter of the cells by adding EDTA solution or the like reagent.

The method may also comprise providing nanofibrillar cellulose hydrogel, which may be for culturing and/or for forming the transplantable cell composition. The cell aggregates may be provided in the hydrogel and/or combined with the hydrogel. The hydrogel may be mixed, preferably by mixing gently with a pipette tip or the like tool, to obtain an even distribution of cells and hydrogel, and to avoid breaking the cells or cell spheroids and to avoid bubble formation.

To obtain the transplantable cells or cell spheroids the cells may be cultured in a cell culture medium which does not contain compounds, which could cause rejection or other immunological reactions in the recipient, or which could provide a risk of disease, such as other type of cell-derived material, and/or which cell culture medium is animal-origin free, xeno-free and/or feeder-free. Such medium preferably contains only the essential cell culture compounds needed for desired cell culture, such as stem cell culture. It is also possible that during the cell culturing the culture medium may be changed into such medium in a last step, i.e. before providing or transferring the cells to the final nanocellulosic matrix.

Alternatively, or in addition, the cells may be washed after obtaining from a cell culture, for example by using a suitable buffer medium or solution, and/or any suitable animal origin free, xeno-free and/or feeder-free medium or solution. The washing medium does not contain undesired compounds which could cause problems in the transplantation, such as compounds derived from other types of cells, for example other types of cells, such as other animal cells or microbial cells. In some cases such compounds may be present in a cell culture medium used for culturing the cells. In general an aqueous medium containing only buffering compounds and optionally salts, surfactants, plasticizers, emulsifiers and the like compounds may be used in the transplantable composition. The salts, buffers and the like agents may be provided to obtain suitable physiological conditions for the cells. One example of such medium is a buffer solution, especially buffer-salt-solution, for example isotonic buffer, such as phosphate buffered saline. At the simplest the buffer solution contains only one or more buffering agent(s) and optionally one or more salt(s). The buffer solution may also contain one or more osmotic/oncotic stabilizer(s), free radical scavenger(s)/antioxidant(s), ion chelator(s), membrane stabilizer(s) and/or energy substrate(s). The medium may consist of an aqueous solution of the ingredients disclosed herein. In general, a buffer solution is an aqueous solution comprising a mixture of weak acid and its conjugate base, or vice versa. Buffer solution may be used to maintain pH at substantially or nearly constant value. The pH of the buffer solution may be in the range of 6-8, such as 7-8, for example 7.0-7.7. Especially stem cells may require a pH range around 7.4, such as 7.2-7.6.

Examples of buffering agents useful in biological applications include TAPS ([Tris(hydroxymethyl)methylamino]propanesulfonic acid), bicine (2-(bis(2-hydroxyethyl)amino)acetic acid), Tris(tris(hydroxymethyl)aminomethane) or, (2-Amino-2-(hydroxymethyl)propane-1,3-diol), tricine (3-[N-Tris(hydroxymethyl)methylamino]-2-hydroxypropanesulfonic acid), TAPSO (3-[N-Tris(hydroxymethyl)methylamino]-2-hydroxypropanesulfonic acid), HEPES (4-(2-hydroxyethyl)-1-piperazineethanesulfonic acid), TES (2-[[1,3-dihydroxy-2-(hydroxymethyl)propan-2-yl]amino]ethanesulfonic acid), MOPS (3-(N-morpholino)propanesulfonic acid), PIPES (piperazine-N,N′-bis(2-ethanesulfonic acid)), cacodylate (dimethylarsenic acid), and MES (2-(N-morphol ino)ethanesulfonic acid).

One specific example of a general buffer solution is phosphate buffered saline (PBS), which usually has a pH of about 7.4. It is a water-based salt solution containing disodium hydrogen phosphate, sodium chloride and, in some formulations, potassium chloride and potassium dihydrogen phosphate. The osmolarity and ion concentrations of the solutions match those of the human body, so it is isotonic.

In general the buffer solution comprises one or more buffering agent(s). In one example the buffer solution comprises zwitterionic buffering agent, such as 4-(2-hydroxyethyl)-1-piperazineethanesulfonic acid (HEPES). The buffering agent(s) should preferably have a pKa value in the range of 6-8. The buffering agent content in the buffer solution may be less than 100 mM, such as 10-50 mM, or 20-30 mM, for example 20-25 mM. In one example the buffer is HEPES buffer solution (such as 10 mM HEPES, 150 mM NaCl, 10 mM EDTA, pH 7.4)

In one embodiment the method comprises removing cell culture medium before providing the cells or cell aggregates in the nanofibrillar cellulose hydrogel and/or combining the cells or cell aggregates with a nanofibrillar cellulose hydrogel, such as by filtering, centrifuging and/or washing the cells or cell aggregates with a medium containing no other type of cell-derived material and/or which is animal-origin free, xeno-free and/or feeder-free. More particularly, the cells or cell aggregates may be filtered and/or centrifuged to remove the undesired medium and optionally other material. The filtering may be aided with vacuum.

The formed cell aggregates, which are multicellular aggregates, are herein called cell spheroids. The culturing may be carried out for at least three days, such as for 3-14 days, or for 3-7 days. Especially the method is suitable for stem cells. After the formation of aggregates, the cells may be harvested and washed with a suitable medium or buffer and/or suspended in to a suitable medium or buffer, such as a buffer or medium having a pH in the range of 6-8 as disclosed in previous. After this the cell aggregates may be transferred to the NFC hydrogel. The NFC hydrogel will form an interstitial matrix between the cell aggregates, which matrix reinforces the structure, immobilizes the cells but enables flow of agents though the matrix, which enables for example cell signalling, flow of nutrients and other important functions. The matrix of the embodiments refers to a matrix surrounding cells and forming a porous three-dimensional lattice, which is functionally similar to the interstitial matrix found in extracellular matrices is tissues. The matrix may be evenly distributed in the composition and/or between the cells. The matrix may be formed and/or further developed, i.e. the NFC may react with the cells to form the matrix, after the cells have been present in the matrix for a suitable period of time, such as at least for 6 hours, at least for 12 hours, or at least for 24 hours. The cells may be stored and/or incubated in the nanofibrillar cellulose hydrogel for several days or even for weeks before use.

In another example the cells are cultured in nanofibrillar cellulose hydrogel, which may contain cell culture medium. The cells may be harvested and transferred to another nanofibrillar cellulose hydrogel, or they may be provided to the transplantation in the same hydrogel wherein they were cultured. In such case it may be necessary to wash the cells and/or to adjust the concentration of the hydrogel into a desired range. In such case the cells are provided in the nanofibrillar cellulose hydrogel to obtain a transplantable cell composition comprising eukaryotic cells in a nanofibrillar cellulose hydrogel, and there is no need to specifically transfer the cells from a different source.

It was found out that nanofibrillar cellulose, either chemically non-modified or chemically modified, especially anionically modified, promoted the formation of cell spheroids and stabilized them. The cell spheroids could be used for transplantation and the like methods involving administering the cells to a subject, for example by injecting or implanting. The NFC hydrogel used especially with stem-cell spheroids reduced further agglomeration of the cells and insulated cells from injection shearing forces during cell transplantation. The cells could differentiate to desired cell types in the NFC hydrogel after transplantation.

The concentration of the nanofibrillar cellulose in the hydrogel, preferably in the final transplantable composition, may in the range of 1-3%, which is suitable for injectable compositions. The concentration may be in the range of 1-2.5% (w/w), or 1.3-2.2% (w/w), for example 1.5-2.0% (w/w). These concentrations were found to facilitate maintaining the cell spheroids in desired and/or obtained size and shape. It was found out that the concentration should not be lower as the viscosity of the hydrogel may be too low in concentration below 1% (w/w), or below 1.5% (w/w). On the other hand, the concentration shall not be too high, such as over 5% (w/w), or over 3% (w/w) or over 2.5% (w/w), because the flow of agents in the hydrogel matrix may decrease or even may be blocked. Too high concentration may also interfere the cell spheroids. The cell density in the transplantable cell composition may be in the range of 1×10⁴ cells/ml-1×10⁸ cells/ml, such as in the range of 1×10⁵ cells/ml-1×10⁷ cells/ml.

The nanofibrillar cellulose should have adequate degree of fibrillation so that the desired properties and effects are obtained. In one embodiment the nanofibrillar cellulose has an average diameter of a fibril in the range of 1-200 nm and/or, when dispersed in water, provides a storage modulus of 350 Pa or more, such as in the range of 350-5000 Pa, or preferably 350-1000 Pa, and yield stress of 25 Pa or more, such as in the range of 25-300 Pa, preferably 25-75 Pa, determined by stress controlled rotational rheometer with gradually increasing shear stress in a range of 0.001-100 Pa at a frequency 10 rad/s, strain 2%, at 25° C.

The nanofibrillar cellulose may be the only matrix material in the composition, such as the only polymeric material in the composition. However, it is also possible to include other polymeric materials in addition to NFC, such as hyaluronan, hyaluronic acid and its derivates, peptide-based materials, proteins, other polysaccharides e.g. alginate, polyethylene glycol. Compositions forming a semi-interpenetrating network (semi-IPN) may be obtained, where nanofibrillar cellulose provides structural stability. The content of the other polymeric materials in the total composition as dry weight may be in the range of 20-80% (w/W), such as 40-60% (w/W), or 10-30% (w/w) or 10-20% (w/w).

The cells and cell spheroids can be studied in the NFC hydrogel visually, for example microscopically, because of the optical properties of the hydrogel. Other tests can be also carried out while the cells are in the hydrogel matrix, as the matrix allows flow of molecular substances. The cells or cell spheroids can be released from the NFC hydrogel by degrading the hydrogel enzymatically, for example by using one or more cellulase enzymes.

The term “cell culture” or “culturing of cells” refers to maintaining, transporting, isolating, culturing, propagating, passaging and/or differentiating of cells or tissues. Cells may be in any arrangement for example as individual cells, monolayers, cell clusters or spheroids or as a tissue.

### Cells

In the present methods and products, cells are provided. The cells may be eukaryotic cells. Eukaryotic cells may be plant cells, yeast cells or animal cells. Examples of eukaryotic cells include transplantable cells, such as stem cells. In the present methods the cells are preferably animal cells or human cells. The cells may be present as aggregates as explained in previous.

Specific examples of cells include stem cells, undifferentiated cells, precursor cells, as well as fully differentiated cells and combinations thereof. In some examples the cells comprise cell types selected from the group consisting of keratocytes, keratinocytes, fibroblast cells, epithelial cells and combinations thereof. In some examples the cells are selected from the group consisting of stem cells, progenitor cells, precursor cells, connective tissue cells, epithelial cells, muscle cells, neuronal cells, endothelial cells, fibroblasts, keratinocytes, smooth muscle cells, stromal cells, mesenchymal cells, immune system cells, hematopoietic cells, dendritic cells, hair follicle cells and combinations thereof. The cells may be genetically modified cells, such as transgenic cells, cisgenic cells or knock-out cells, or pathogenic cells. Such cells may be used for example for drug research or in therapy. Especially stem cells may be used in therapeutical applications, for example provided to a patient.

Eukaryotic cells may be mammalian cells. Examples of mammalian cells include human cells, mouse cells, rat cells, rabbit cells, monkey cells, pig cells, bovine cells, chicken cells and the like.

In one embodiment the cells are stem cells, such as omnipotent, pluripotent, multipotent, oligopotent or unipotent stem cells. Stem cells are cells capable of renewing themselves through cell division and can differentiate into multi-lineage cells. These cells may be categorized as embryonic stem cells (ESCs), induced pluripotent stem cells (iPSCs), and adult stem cells, also called as tissue-specific or somatic stem cells. The stem cells may be human stem cells, which may be of non-embryonic origin, such as adult stem cells. These are undifferentiated cells found throughout the body after differentiation. They are responsible for e.g. organ regeneration and capable of dividing in pluripotent or multipotent state and differentiating into differentiated cell lineages. The stem cells may be human embryonic stem cell lines generated without embryo destruction, such as described for example in Cell Stem Cell. 2008 Feb. 7; 2(2):113-7. The stem cells may be obtained from a source of autologous adult stem cells, such as bone marrow, adipose tissue, or blood.

Examples of stem cells include mesenchymal stem cells (MSC), multipotent adult progenitor cells (MAPC®), induced pluripotent stem cells (iPS), and hematopoietic stem cells.

In case of human stem cells the cells may be non-embryonic cells or embryonic cells, such as hESCs (human embryonic stem cells), which have been derived without destroying the embryo. In case of human embryonic stem cells the cells may be from a deposited cell line or made from unfertilized eggs, i.e. “parthenote” eggs or from parthenogenetically activated ovum, so that no human embryos are destroyed.

In one embodiment the cells are mesenchymal stem cells (MSC). Mesenchymal stem cells (MSCs) are adult stem cells which can be isolated from human and animal sources, such as from mammals. Mesenchymal stem cells are multipotent stromal cells that can differentiate into a variety of cell types, including osteoblasts, chondrocytes, myocytes and adipocytes. Mesenchyme itself is embryonic connective tissue that is derived from the mesoderm and that differentiates into hematopoietic and connective tissue. However mesenchymal stem cells do not differentiate into hematopoietic cells. The terms mesenchymal stem cell and marrow stromal cell have been used interchangeably for many years, but neither term is sufficiently descriptive. Stromal cells are connective tissue cells that form the supportive structure in which the functional cells of the tissue reside. While this is an accurate description for one function of MSCs, the term fails to convey the relatively recently discovered roles of MSCs in the repair of tissue. The term encompasses multipotent cells derived from other non-marrow tissues, such as placenta, umbilical cord blood, adipose tissue, adult muscle, corneal stroma or the dental pulp of deciduous baby teeth. The cells do not have the capacity to reconstitute an entire organ

The International Society for Cellular Therapy has proposed minimum criteria to define MSCs. These cells (a) should exhibits plastic adherence, (b) possess specific set of cell surface markers, i.e. cluster of differentiation (CD)73, D90, CD105 and lack expression of CD14, CD34, CD45 and human leucocyte antigen-DR (HLA-DR) and (c) have the ability to differentiate in vitro into adipocyte, chondrocyte and osteoblast. These characteristics are valid for all MSCs, although few differences exist in MSCs isolated from various tissue origins. MSCs are present not only in fetal tissues but also in many adult tissues with few exceptions. Efficient population of MSCs has been reported from bone marrow. Cells which exhibits characteristics of MSCs have been isolated from adipose tissue, amniotic fluid, amniotic membrane, dental tissues, endometrium, limb bud, menstrual blood, peripheral blood, placenta and fetal membrane, salivary gland, skin and foreskin, sub-amniotic umbilical cord lining membrane, synovial fluid and Wharton's jelly.

Human mesenchymal stem cells (hMSC) display a very high degree of plasticity and are found in virtually all organs with the highest density in bone marrow. hMSCs serve as renewable source for mesenchymal cells and have pluripotent ability of differentiating into several cell lineages, including osteoblasts, chondrocytes, adipocytes, skeletal and cardiac myocytes, endothelial cells, and neurons in vitro upon appropriate stimulation, and in vivo after transplantation.

In one example the cells are multipotent adult progenitor cells (MAPC), which are derived from a primitive cell population that can be harvested from bone marrow, muscle and brain. MAPC are a more primitive cell population than mesenchymal stem cells, whilst they imitate embryonic stem cells characteristics they still retain adult stem cells potential in cell therapy. In vitro, MAPC demonstrated a vast differentiation potential to adipogenic, osteogenic, neurogenic, hepatogenic, hematopoietic, myogenic, chondrogenic, epithelial, and endothelial lineages. A key feature of MAPC is that they show large proliferative potential in vitro without losing their phenotype. MAPC may be used for treating a variety of diseases such as ischaemic stroke, graft versus host disease, acute myocardial infarct, organ transplant, bone repair and myelodysplasia. MAPC also enhance bone formation, promote neovascularisation, and have immunomodulatory effects.

Induced pluripotent stem cells (iPS) are a type of pluripotent stem cell that can be generated directly from adult cells. They can propagate practically indefinitely and may give rise to every other cell type in the body, including neurons, heart, pancreatic and liver cells. Induced pluripotent stem cells can be derived directly from adult tissues and they can be made in a patient-matched manner so they may be provided a transplants without the risk of immune rejection. Human induced pluripotent stem cells are of special interest, and they can be generated from for example human fibroblasts, keratinocytes, peripheral blood cells, renal epithelial cells or other suitable cell types.

Hematopoietic stem cells (HSCs), also called as blood stem cells, are cells that can develop into all types of blood cells, including white blood cells, red blood cells, and platelets. Hematopoietic stem cells are found in the peripheral blood and the bone marrow. HSCs give rise to both the myeloid and lymphoid lineages of blood cells. Myeloid and lymphoid lineages both are involved in dendritic cell formation. Myeloid cells include monocytes, macrophages, neutrophils, basophils, eosinophils, erythrocytes, and megakaryocytes to platelets. Lymphoid cells include T cells, B cells, and natural killer cells. Hematopoietic stem cell transplants can be used in the treatment of cancers and other immune system disorders.

In general the cells may be cultured in a hydrogel, and they may be also stored in it. The cells can be maintained and proliferated on or in the hydrogel without animal or human based agents or medium originating outside the cells. The cells may be evenly dispersed on or in the hydrogel.

Initially the cells may be pre-cultured in a separate culture, and recovered and transferred into a new medium, which may be similar or different than the culture medium. A cell suspension is obtained. This, or another cell suspension, may be combined and/or mixed with the nanofibrillar cellulose, such as a hydrogel comprising nanofibrillar cellulose, to obtain or form a cell system or cell composition. If cells are cultured in the cell system a cell culture is formed. The cell system or culture may be 2D system or culture or a 3D system or culture. 2D system or culture refers to a system or culture in a membrane and/or as a layer. 3D system or culture refers to a system or culture in the nanofibrillar cellulose, wherein the cells are permitted to grow and/or interact in all three dimensions. The NFC hydrogel matrix mimics the natural extracellular matrix structure and provides efficient transport of nutrients, gases and the like. In one example the cell system is a 3D cell system.

### Nanofibrillar Cellulose

The starting material for forming the hydrogel is nanofibrillar cellulose, also called as nanocellulose, which refers to isolated cellulose fibrils or fibril bundles derived from cellulose raw material. Nanofibrillar cellulose is based on a natural polymer that is abundant in nature. Nanofibrillar cellulose has a capability of forming viscous hydrogel in water. Nanofibrillar cellulose production techniques may be based on disintegrating fibrous raw material, such as grinding of aqueous dispersion of pulp fibers to obtain nanofibrillated cellulose. After the grinding or homogenization process, the obtained nanofibrillar cellulose material is a dilute viscoelastic hydrogel.

The obtained material usually exists at a relatively low concentration homogeneously distributed in water due to the disintegration conditions. The starting material may be an aqueous gel at a concentration of 0.2-10% (w/w), for example 0.2-5% (w/w). The nanofibrillar cellulose may be obtained directly from the disintegration of fibrous raw material. An example of commercially available nanofibrillar cellulose hydrogel is GrowDex® by UPM.

Because of its nanoscale structure nanofibrillar cellulose has unique properties which enable functionalities which cannot be provided by conventional non-nanofibrillar cellulose. It is possible to prepare materials and products which exhibit different properties than conventional products or products using conventional cellulosic materials. However, because of the nanoscale structure nanofibrillar cellulose is also a challenging material. For example dewatering or handling of nanofibrillar cellulose may be difficult.

The nanofibrillar cellulose may be prepared from cellulose raw material of plant origin, or it may also be derived from certain bacterial fermentation processes. The nanofibrillar cellulose is preferably made of plant material. The raw material may be based on any plant material that contains cellulose. In one example the fibrils are obtained from non-parenchymal plant material. In such case the fibrils may be obtained from secondary cell walls. One abundant source of such cellulose fibrils is wood fibres. The nanofibrillar cellulose may be manufactured by homogenizing wood-derived fibrous raw material, which may be chemical pulp. Cellulose fibers are disintegrated to produce fibrils which have an average diameter of only some nanometers, which may be 200 nm or less in most cases, and gives a dispersion of fibrils in water. The fibrils originating from secondary cell walls are essentially crystalline with degree of crystallinity of at least 55%. Such fibrils may have different properties than fibrils originated from primary cell walls, for example the dewatering of fibrils originating from secondary cell walls may be more challenging. In general in the cellulose sources from primary cell walls, such as sugar beet, potato tuber and banana rachis, the microfibrils are easier to liberate from the fibre matrix than fibrils from wood, and the disintegration requires less energy. However, these materials are still somewhat heterogeneous and consist of large fibril bundles.

Non-wood material may be from agricultural residues, grasses or other plant substances such as straw, leaves, bark, seeds, hulls, flowers, vegetables or fruits from cotton, corn, wheat, oat, rye, barley, rice, flax, hemp, manila hemp, sisal hemp, jute, ramie, kenaf, bagasse, bamboo or reed. The cellulose raw material could be also derived from the cellulose-producing micro-organism. The micro-organisms can be of the genus Acetobacter, Agrobacterium, Rhizobium, Pseudomonas or Alcaligenes, preferably of the genus Acetobacter and more preferably of the species Acetobacter xylinumor or Acetobacter pasteurianus.

It was found out that nanofibrillar cellulose obtained from wood cellulose is preferable for medical or scientific products described herein. Wood cellulose is available in large amounts, and the preparation methods developed for wood cellulose enable producing nanofibrillar materials suitable for the products. The nanofibrillar cellulose obtained by fibrillating plant fibers, especially wood fibers, differs structurally from nanofibrillar cellulose obtained from microbes, and it has different properties. For example compared to bacterial cellulose, nanofibrillated wood cellulose is homogenous and more porous and loose material, which is advantageous in applications involving living cells. Bacterial cellulose is usually used as such without similar fibrillation as in plant cellulose, so the material is different also in this respect. Bacterial cellulose is dense material which easily forms small spheroids and therefore the structure of the material is discontinuous, and it is not desired to use such material in the applications relating to living cells, especially when homogeneity of the material is required.

Wood may be from softwood tree such as spruce, pine, fir, larch, douglas-fir or hemlock, or from hardwood tree such as birch, aspen, poplar, alder, eucalyptus, oak, beech or acacia, or from a mixture of softwoods and hardwoods. In one example the nanofibrillar cellulose is obtained from wood pulp. The nanofibrillar cellulose may be obtained from hardwood pulp. In one example the hardwood is birch. The nanofibrillar cellulose may be obtained from softwood pulp. In one example said wood pulp is chemical pulp. Chemical pulp may be desired for the products disclosed herein. Chemical pulp is pure material and may be used in a wide variety of applications. For example chemical pulp lack the pitch and resin acids present in mechanical pulp, and it is more sterile or easily sterilisable. Further, chemical pulp is more flexible and provides advantageous properties for example in medical and scientific materials. For example very homogenous nanofibrillar cellulose materials may be prepared without excess processing or need for specific equipment or laborious process steps.

Nanofibrillar cellulose, including the cellulose fibrils and/or fibril bundles, is characterized by a high aspect ratio (length/diameter). The average length of nanofibrillar cellulose (the median length of particles such as fibrils or fibril bundles) may exceed 1 μm, and in most cases it is 50 μm or less. If the elementary fibrils are not completely separated from each other, the entangled fibrils may have an average total length for example in the range of 1-100 μm, 1-50 μm, or 1-20 μm. However, if the nanofibrillar material is highly fibrillated, the elementary fibrils may be completely or almost completely separated and the average fibril length is shorter, such as in the range of 1-10 μm or 1-5 μm. This applies especially for native grades of fibrils which are not shortened or digested, for example chemically, enzymatically or mechanically. However, strongly derivatized nanofibrillar cellulose may have a shorter average fibril length, such as in the range of 0.3-50 μm, such as 0.3-20 μm, for example 0.5-10 μm or 1-10 μm. Especially shortened fibrils, such as enzymatically or chemically digested fibrils, or mechanically treated material, may have an average fibril length of less than 1 μm, such as 0.1-1 μm, 0.2-0.8 μm or 0.4-0.6 μm. The fibril length and/or diameter may be estimated microscopically, for example using CRYO-TEM, SEM or AFM images.

The average diameter (width) of nanofibrillar cellulose is less than 1 μm, or 500 nm or less, such as in the range of 1-500 nm, but preferably 200 nm or less, even 100 nm or less or 50 nm or less, such as in the range of 1-200 nm, 2-200 nm, 2-100 nm, or 2-50 nm, even 2-20 for highly fibrillated material. The diameters disclosed herein may refer to fibrils and/or fibril bundles. The smallest fibrils are in the scale of elementary fibrils, the average diameter being typically in the range of 2-12 nm. The dimensions and size distribution of the fibrils depend on the refining method and efficiency. In case of highly refined native nanofibrillar cellulose, the average fibril diameter, including fibril bundles, may be in the range of 2-200 nm or 5-100 nm, for example in the range of 10-50 nm. Nanofibrillar cellulose is characterized by a large specific surface area and a strong ability to form hydrogen bonds. In water dispersion, the nanofibrillar cellulose typically appears as either light or turbid gel-like material. Depending on the fiber raw material, nanofibrillar cellulose obtained from plants, especially wood, may also contain small amounts of other plant components, especially wood components, such as hemicellulose or lignin. The amount is dependent on the plant source.

In general cellulose nanomaterials may be divided into categories according to TAPPI W13021, which provides standard terms for cellulose nanomaterials. Not all of these materials are nanofibrillar cellulose. Two main categories are “Nano objects” and “Nano structured materials”. Nanostructured materials include “Cellulose microcrystals” (sometimes called as CMC) having a diameter of 10-12 μm and length:diameter ratio (L/D)<2, and “Cellulose microfibrils” having a diameter of 10-100 nm and a length of 0.5-50 μm. Nano objects include “Cellulose nanofibers”, which can be divided into “Cellulose nanocrystals” (CNC) having a diameter of 3-10 nm and L/D>5, and “Cellulose nanofibrils” (CNF or NFC), having a diameter of 5-30 nm and L/D>50.

Different grades of nanofibrillar cellulose may be categorized based on three main properties: (i) size distribution, length and diameter (ii) chemical composition, and (iii) rheological properties. To fully describe a grade, the properties may be used in parallel. Examples of different grades include native (chemically and/or enzymatically unmodified) NFC, oxidized NFC (high viscosity), oxidized NFC (low viscosity), carboxymethylated NFC and cationized NFC. Within these main grades, also sub-grades exist, for example: extremely well fibrillated vs. moderately fibrillated, high degree of substitution vs. low degree of substitution, low viscosity vs. high viscosity etc. The fibrillation technique and the chemical pre-modification have an influence on the fibril size distribution. Typically, non-ionic grades have wider average fibril diameter (for example in the range of 10-100 nm, or 10-50 nm) while the chemically modified grades are a lot thinner (for example in the range of 2-20 nm). Distribution is also narrower for the modified grades. Certain modifications, especially TEMPO-oxidation, yield shorter fibrils.

Depending on the raw material source, e.g. hardwood vs. softwood pulp, different polysaccharide composition exists in the final nanofibrillar cellulose product. Commonly, the non-ionic grades are prepared from bleached birch pulp, which yields high xylene content (25% by weight). Modified grades are prepared either from hardwood or softwood pulps. In those modified grades, the hemicelluloses are also modified together with the cellulose domain. Most probably, the modification is not homogeneous, i.e. some parts are more modified than others. Thus, detailed chemical analysis is usually not possible as the modified products are complicated mixtures of different polysaccharide structures.

In an aqueous environment, a dispersion of cellulose nanofibrils forms a viscoelastic hydrogel network. The gel is formed already at relatively low concentrations of for example 0.05-0.2% (w/w) by dispersed and hydrated entangled fibrils. The viscoelasticity of the NFC hydrogel may be characterized for example with dynamic oscillatory rheological measurements.

The nanofibrillar cellulose hydrogels exhibit characteristic rheological properties. For example they are shear-thinning or pseudoplastic materials, which may be considered as a special case of thixotropic behavior, which means that their viscosity depends on the speed or force by which the material is deformed. When measuring the viscosity in a rotational rheometer, the shear-thinning behavior is seen as a decrease in viscosity with increasing shear rate. The hydrogels show plastic behavior, which means that a certain shear stress (force) is required before the material starts to flow readily. This critical shear stress is often called the yield stress. The yield stress can be determined from a steady state flow curve measured with a stress controlled rheometer. When the viscosity is plotted as function of applied shear stress, a dramatic decrease in viscosity is seen after exceeding the critical shear stress. The zero shear viscosity and the yield stress are the most important rheological parameters to describe the suspending power of the materials. These two parameters separate the different grades quite clearly and thus enable classification of the grades.

The dimensions of the fibrils or fibril bundles are dependent for example on the raw material, the disintegration method and number of disintegration runs. Mechanical disintegration of the cellulose raw material may be carried out with any suitable equipment such as a refiner, grinder, disperser, homogenizer, colloider, friction grinder, pin mill, rotor-rotor disperser, ultrasound sonicator, fluidizer such as microfluidizer, macrofluidizer or fluidizer-type homogenizer. The disintegration treatment is performed at conditions wherein water is sufficiently present to prevent the formation of bonds between the fibers.

In one example the disintegration is carried out by using a disperser having at least one rotor, blade or similar moving mechanical member, such as a rotor-rotor disperser, which has at least two rotors. In a disperser the fiber material in dispersion is repeatedly impacted by blades or ribs of rotors striking it from opposite directions when the blades rotate at the rotating speed and at the peripheral speed determined by the radius (distance to the rotation axis) in opposite directions. Because the fiber material is transferred outwards in the radial direction, it crashes onto the wide surfaces of the blades, i.e. ribs, coming one after the other at a high peripheral speed from opposite directions; in other words, it receives a plurality of successive impacts from opposite directions. Also, at the edges of the wide surfaces of the blades, i.e. ribs, which edges form a blade gap with the opposite edge of the next rotor blade, shear forces occur, which contribute to the disintegration of the fibers and detachment of fibrils. The impact frequency is determined by the rotation speed of the rotors, the number of the rotors, the number of blades in each rotor, and the flow rate of the dispersion through the device.

In a rotor-rotor disperser the fiber material is introduced through counter-rotating rotors, outwards in the radial direction with respect to the axis of rotation of the rotors in such a way that the material is repeatedly subjected to shear and impact forces by the effect of the different counter-rotating rotors, whereby it is simultaneously fibrillated. One example of a rotor-rotor disperser is an Atrex device.

Another example of a device suitable for disintegrating is a pin mill, such as a multi-peripheral pin mill. One example of such device includes a housing and in it a first rotor equipped with collision surfaces; a second rotor concentric with the first rotor and equipped with collision surfaces, the second rotor being arranged to rotate in a direction opposite to the first rotor; or a stator concentric with the first rotor and equipped with collision surfaces. The device includes a feed orifice in the housing and opening to the center of the rotors or the rotor and stator, and a discharge orifice on the housing wall and opening to the periphery of the outermost rotor or stator.

In one example the disintegrating is carried out by using a homogenizer. In a homogenizer the fiber material is subjected to homogenization by an effect of pressure. The homogenization of the fiber material dispersion to nanofibrillar cellulose is caused by forced through-flow of the dispersion, which disintegrates the material to fibrils. The fiber material dispersion is passed at a given pressure through a narrow through-flow gap where an increase in the linear velocity of the dispersion causes shearing and impact forces on the dispersion, resulting in the removal of fibrils from the fiber material. The fiber fragments are disintegrated into fibrils in the fibrillating step.

As used herein, the term “fibrillation” generally refers to disintegrating fiber material mechanically by work applied to the particles, where cellulose fibrils are detached from the fibers or fiber fragments. The work may be based on various effects, like grinding, crushing or shearing, or a combination of these, or another corresponding action that reduces the particle size. The expressions “disintegration” or “disintegration treatment” may be used interchangeably with “fibrillation”.

The fiber material dispersion that is subjected to fibrillation is a mixture of fiber material and water, also herein called “pulp”. The fiber material dispersion may refer generally to whole fibers, parts (fragments) separated from them, fibril bundles, or fibrils mixed with water, and typically the aqueous fiber material dispersion is a mixture of such elements, in which the ratios between the components are dependent on the degree of processing or on the treatment stage, for example number of runs or “passes” through the treatment of the same batch of fiber material.

One way to characterize the nanofibrillar cellulose is to use the viscosity of an aqueous solution containing said nanofibrillar cellulose. The viscosity may be for example Brookfield viscosity or zero shear viscosity. The specific viscosity, as described herein, distinguishes nanofibrillar cellulose from non-nanofibrillar cellulose.

In one example the apparent viscosity of the nanofibrillar cellulose is measured with a Brookfield viscometer (Brookfield viscosity) or another corresponding apparatus. Suitably a vane spindle (number 73) is used. There are several commercial Brookfield viscometers available for measuring apparent viscosity, which all are based on the same principle. Suitably RVDV spring (Brookfield RVDV-III) is used in the apparatus. A sample of the nanofibrillar cellulose is diluted to a concentration of 0.8% by weight in water and mixed for 10 min. The diluted sample mass is added to a 250 ml beaker and the temperature is adjusted to 20° C.±1° C., heated if necessary and mixed. A low rotational speed 10 rpm is used. In general Brookfield viscosity may be measured at 20° C.±1° C., at a consistency of 0.8% (w/w) and at 10 rpm.

The nanofibrillar cellulose, for example provided as a starting material in the method, may be characterized by the viscosity it provides in a water solution. The viscosity describes, for example, the fibrillation degree of the nanofibrillar cellulose. In one example the nanofibrillar cellulose when dispersed in water provides a Brookfield viscosity of at least 2000 mPa·s, such as at least 3000 mPa·s, measured at 20° C.±1° C., at a consistency of 0.8% (w/w) and at 10 rpm. In one example the nanofibrillar cellulose, when dispersed in water, provides a Brookfield viscosity of at least 10000 mPa·s measured at 20° C.±1° C., at a consistency of 0.8% (w/w) and at 10 rpm. In one example the nanofibrillar cellulose, when dispersed in water, provides a Brookfield viscosity of at least 15000 mPa·s measured at 20° C.±1° C., at a consistency of 0.8% (w/w) and at 10 rpm. Examples of Brookfield viscosity ranges of said nanofibrillar cellulose when dispersed in water include 2000-20000 mPa·s, 3000-20000 mPa·s, 10000-20000 mPa·s, 15000-20000 mPa·s, 2000-25000 mPa·s, 3000-25000 mPa·s, 10000-25000 mPa·s, 15000-25000 mPa·s, 2000-30000 mPa·s, 3000-30000 mPa·s, 10000-30000 mPa·s, and 15000-30000 mPa·s, measured at 20° C.±1° C., at a consistency of 0.8% (w/w) and at 10 rpm.

The nanofibrillar cellulose may also be characterized by the average diameter (or width), or by the average diameter together with the viscosity, such as Brookfield viscosity or zero shear viscosity. In one example nanofibrillar cellulose suitable for use in the products described herein has an average fibril diameter in the range of 1-200 nm, or 1-100 nm. In one example said nanofibrillar cellulose has an average fibril diameter in the range of 1-50 nm, such as 2-20 nm or 5-30 nm. In one example said nanofibrillar cellulose has an average fibril diameter in the range of 2-15 nm, such as in the case of TEMPO oxidized nanofibrillar cellulose.

The diameter of a fibril may be determined with several techniques, such as by microscopy. Fibril thickness and width distribution may be measured by image analysis of the images from a field emission scanning electron microscope (FE-SEM), a transmission electron microscope (TEM), such as a cryogenic transmission electron microscope (cryo-TEM), or an atomic force microscope (AFM). In general AFM and TEM suit best for nanofibrillar cellulose grades with narrow fibril diameter distribution.

A rheometer viscosity of the nanofibrillar cellulose dispersion may be measured according to one example at 22° C. with a stress controlled rotational rheometer (AR-G2, TA Instruments, UK) equipped with a narrow gap vane geometry (diameter 28 mm, length 42 mm) in a cylindrical sample cup having a diameter of 30 mm. After loading the samples to the rheometer they are allowed to rest for 5 min before the measurement is started. The steady state viscosity is measured with a gradually increasing shear stress (proportional to applied torque) and the shear rate (proportional to angular velocity) is measured. The reported viscosity (=shear stress/shear rate) at a certain shear stress is recorded after reaching a constant shear rate or after a maximum time of 2 min. The measurement is stopped when a shear rate of 1000 s⁻¹ is exceeded. This method may be used for determining the zero-shear viscosity.

In another example rheological measurements of the hydrogel samples were carried out with a stress controlled rotational rheometer (AR-G2, TA instruments, UK) equipped with 20 mm plate geometry. After loading the samples to the rheometer, 1 mm gap, without dilution, they were allowed to settle for 5 min before the measurement was started. The stress sweep viscosity was measured with gradually increasing shear stress in a range of 0,001-100 Pa at the frequency 10 rad/s, strain 2%, at 25° C. Storage modulus, loss modulus and yield stress/fracture strength can be determined.

It was found out that there is a minimum viscosity level require for hydrogel to retain its shape after the injection. This may be characterized by storage modulus of 350 Pa or more, and yield stress/fracture strength of 25 Pa or more.

In one example the nanofibrillar cellulose, for example provided as a starting material in the method, when dispersed in water, provides a zero shear viscosity (“plateau” of constant viscosity at small shearing stresses) in the range of 1000-100000 Pa·s, such as in the range of 5000-50000 Pa·s, and a yield stress (shear stress where the shear thinning begins) in the range of 1-50 Pa, such as in the range of 3-15 Pa, determined by rotational rheometer at a consistency of 0.5% (w/w) by weight in aqueous medium at 22° C.±1° C. Such nanofibrillar cellulose may also have an average fibril diameter of 200 nm or less, such as in the range of 1-200 nm.

Turbidity is the cloudiness or haziness of a fluid caused by individual particles (total suspended or dissolved solids) that are generally invisible to the naked eye. There are several practical ways of measuring turbidity, the most direct being some measure of attenuation (that is, reduction in strength) of light as it passes through a sample column of water. The alternatively used Jackson Candle method (units: Jackson Turbidity Unit or JTU) is essentially the inverse measure of the length of a column of water needed to completely obscure a candle flame viewed through it.

Turbidity may be measured quantitatively using optical turbidity measuring instruments. There are several commercial turbidometers available for measuring turbidity quantitatively. In the present case the method based on nephelometry is used. The units of turbidity from a calibrated nephelometer are called Nephelometric Turbidity Units (NTU). The measuring apparatus (turbidometer) is calibrated and controlled with standard calibration samples, followed by measuring of the turbidity of the diluted NFC sample.

In one turbidity measurement method, a nanofibrillar cellulose sample is diluted in water, to a concentration below the gel point of said nanofibrillar cellulose, and turbidity of the diluted sample is measured. Said concentration where the turbidity of the nanofibrillar cellulose samples is measured is 0.1%. HACH P2100 Turbidometer with a 50 ml measuring vessel is used for turbidity measurements. The dry matter of the nanofibrillar cellulose sample is determined and 0.5 g of the sample, calculated as dry matter, is loaded in the measuring vessel, which is filled with tap water to 500 g and vigorously mixed by shaking for about 30 s. Without delay the aqueous mixture is divided into 5 measuring vessels, which are inserted in the turbidometer. Three measurements on each vessel are carried out. The mean value and standard deviation are calculated from the obtained results, and the final result is given as NTU units.

One way to characterize nanofibrillar cellulose is to define both the viscosity and the turbidity. Low turbidity refers to small size of the fibrils, such as small diameter, as small fibrils scatter light poorly. In general as the fibrillation degree increases, the viscosity increases and at the same time the turbidity decreases. This happens, however, until a certain point. When the fibrillation is further continued, the fibrils finally begin to break and cannot form a strong network any more. Therefore, after this point, both the turbidity and the viscosity begin to decrease.

In one example the turbidity of anionic nanofibrillar cellulose is lower than 90 NTU, for example from 3 to 90 NTU, such as from 5 to 60, for example 8-40 measured at a consistency of 0.1% (w/w) in aqueous medium, and measured by nephelometry. In one example the turbidity of native nanofibrillar may be even over 200 NTU, for example from 10 to 220 NTU, such as from 20 to 200, for example 50-200 measured at measured at 20° C.±1° C. a consistency of 0.1% (w/w) in aqueous medium, and measured by nephelometry. To characterize the nanofibrillar cellulose these ranges may be combined with the viscosity ranges of the nanofibrillar cellulose, such as zero shear viscosity, storage modulus and/or yield stress.

Nanofibrillar cellulose may be or comprise non-modified nanofibrillar cellulose. The drainage of non-modified nanofibrillar cellulose is significantly faster than for example anionic grade. Non-modified nanofibrillar cellulose generally has a Brookfield viscosity in the range of 2000-10000 mPa·s, measured at 20° C.±1° C., at a consistency of 0.8% (w/w) and at 10 rpm. It is preferred that the nanofibrillar cellulose has a suitable carboxylic acid content, such as in the range of 0.6-1.4 mmol COOH/g, for example in the range of 0.7-1.2 mmol COOH/g, or in the range of 0.7-1.0 mmol COOH/g or 0.8-1.2 mmol COOH/g, determined by conductometric titration.

The disintegrated fibrous cellulosic raw material may be modified fibrous raw material. Modified fibrous raw material means raw material where the fibers are affected by the treatment so that cellulose nanofibrils are more easily detachable from the fibers. The modification is usually performed to fibrous cellulosic raw material which exists as a suspension in a liquid, i.e. pulp.

The modification treatment to the fibers may be chemical, enzymatic or physical. In chemical modification the chemical structure of cellulose molecule is changed by chemical reaction (“derivatization” of cellulose), preferably so that the length of the cellulose molecule is not affected but functional groups are added to β-D-glucopyranose units of the polymer. The chemical modification of cellulose takes place at a certain conversion degree, which is dependent on the dosage of reactants and the reaction conditions, and as a rule it is not complete so that the cellulose will stay in solid form as fibrils and does not dissolve in water. In physical modification anionic, cationic, or non-ionic substances or any combination of these are physically adsorbed on cellulose surface.

The cellulose in the fibers may be especially ionically charged after the modification. The ionic charge of the cellulose weakens the internal bonds of the fibers and will later facilitate the disintegration to nanofibrillar cellulose. The ionic charge may be achieved by chemical or physical modification of the cellulose. The fibers may have higher anionic or cationic charge after the modification compared with the starting raw material. Most commonly used chemical modification methods for making an anionic charge are oxidation, where hydroxyl groups are oxidized to aldehydes and carboxyl groups, sulphonization and carboxymethylation. Chemical modifications introducing groups, such as carboxyl groups, which may take part in forming a covalent bond between the nanofibrillar cellulose and the bioactive molecule, may be desired. A cationic charge in turn may be created chemically by cationization by attaching a cationic group to the cellulose, such as quaternary ammonium group.

Nanofibrillar cellulose may comprise chemically modified nanofibrillar cellulose, such as anionically modified nanofibrillar cellulose or cationically modified nanofibrillar cellulose. In one example the nanofibrillar cellulose is anionically modified nanofibrillar cellulose. In one example the anionically modified nanofibrillar cellulose is oxidized nanofibrillar cellulose. In one example the anionically modified nanofibrillar cellulose is sulphonized nanofibrillar cellulose. In one example the anionically modified nanofibrillar cellulose is carboxymethylated nanofibrillar cellulose. The material obtained with the anionical modification of cellulose may be called anionic cellulose, which refers to material wherein the amount or proportion of anionic groups, such as carboxylic groups, is increased by the modification, when compared to a non-modified material. It is also possible to introduce other anionic groups to the cellulose, instead or in addition to carboxylic groups, such as phosphate groups or sulphate groups. The content of these groups may be in the same ranges as is disclosed for carboxylic acid herein.

The cellulose may be oxidized. In the oxidation of cellulose, the primary hydroxyl groups of cellulose may be oxidized catalytically by a heterocyclic nitroxyl compound, such as through N-oxyl mediated catalytic oxidation, for example 2,2,6,6-tetramethylpiperidinyl-1-oxy free radical, generally called “TEMPO”. The primary hydroxyl groups (C6-hydroxyl groups) of the cellulosic β-D-glucopyranose units are selectively oxidized to carboxylic groups. Some aldehyde groups are also formed from the primary hydroxyl groups. Regarding the finding that low degree of oxidation does not allow efficient enough fibrillation and higher degree of oxidation inflicts degradation of cellulose after mechanical disruptive treatment, the cellulose may be oxidized to a level having a carboxylic acid content in the oxidized cellulose in the range of 0.5-2.0 mmol COOH/g pulp, 0.6-1.4 mmol COOH/g pulp, or 0.8-1.2 mmol COOH/g pulp, preferably to 1.0-1.2 mmol COON/g pulp, determined by conductometric titration. When the fibers of oxidized cellulose so obtained are disintegrated in water, they give stable transparent dispersion of individualized cellulose fibrils, which may be, for example, of 3-5 nm in width. With oxidized pulp as the starting medium, it is possible to obtain nanofibrillar cellulose where Brookfield viscosity measured at a consistency of 0.8% (w/w) is at least 10000 mPa·s, for example in the range of 10000-30000 mPa·s.

Whenever the catalyst “TEMPO” is mentioned in this disclosure, it is evident that all measures and operations where “TEMPO” is involved apply equally and analogously to any derivative of TEMPO or any heterocyclic nitroxyl radical capable of catalyzing selectively the oxidation of the hydroxyl groups of C6 carbon in cellulose.

The modifications of nanofibrillar cellulose disclosed herein may also be applied to other fibrillar cellulose grades described herein. For example also highly refined cellulose or microfibrillar cellulose may be similarly chemically or enzymatically modified. However, there are differences for example in the final fibrillation degree of the materials.

In one example such chemically modified nanofibrillar cellulose, when dispersed in water, provides a Brookfield viscosity of at least 10000 mPa·s measured at 20° C.±1° C., at a consistency of 0.8% (w/w) and at 10 rpm. In one example such chemically modified nanofibrillar cellulose, when dispersed in water, provides a Brookfield viscosity of at least 15000 mPa·s measured at 20° C.±1° C., at a consistency of 0.8% (w/w) and at 10 rpm. In one example such chemically modified nanofibrillar cellulose, when dispersed in water, provides a Brookfield viscosity of at least 18000 mPa·s measured at 20° C.±1° C., at a consistency of 0.8% (w/w) and at 10 rpm. Examples of anionic nanofibrillar celluloses used have a Brookfield viscosity in the range of 13000-15000 mPa·s or 18000-20000 mPa·s, or even up to 25000 mPa·s, depending on the degree of fibrillation.

In one example the nanofibrillar cellulose is TEMPO oxidized nanofibrillar cellulose. It provides high viscosity at low concentrations, for example a Brookfield viscosity of at least 20000 mPa·s, even at least 25000 mPa·s, measured at 20° C.±1° C., at a consistency of 0.8% (w/w) and at 10 rpm. In one example the Brookfield viscosity of TEMPO oxidized nanofibrillar cellulose is in the range of 20000-30000 mPa·s, such as 25000-30000 mPa·s, measured at 20° C.±1° C., at a consistency of 0.8% (w/w) and at 10 rpm.

In one example the nanofibrillar cellulose comprises chemically unmodified nanofibrillar cellulose. In one example such chemically unmodified nanofibrillar cellulose, when dispersed in water, provides a Brookfield viscosity of at least 2000 mPa·s, or at least 3000 mPa·s, measured at 20° C.±1° C., at a consistency of 0.8% (w/w) and at 10 rpm.

Auxiliary agents for enhancing the manufacturing process or improving or adjusting the properties of the product may be included in the nanofibrillar cellulose dispersion. Such auxiliary agents may be soluble in the liquid phase of the dispersion, they may form an emulsion or they may be solid. Auxiliary agents may be added already during the manufacturing of the nanofibrillar cellulose dispersion to the raw material or they may be added to a formed nanofibrillar cellulose dispersion or gel. The auxiliary agents may be also added to the final product, for example by impregnating, spraying, dipping, soaking or the like method. The auxiliary agents are usually not covalently bound to the nanofibrillar cellulose, so they may be releasable from the nanocellulose matrix. A controlled and/or sustained release of such agents may be obtained when using NFC as matrix. Examples of auxiliary agents include therapeutic (pharmaceutic) agents and other agents affecting to the properties of the product or to the properties of the active agents, such as buffers, surfactants, plasticizers, emulsifiers or the like. In one example the dispersion contains one or more salts, which may be added to enhance the properties of the final product or to facilitate water removal from the product in the manufacturing process. Examples of salts include chloride salts, such as sodium chloride, calcium chloride and potassium chloride. The salt may be included in an amount in the range of 0.01-1.0% (w/w) of the dry matter in the dispersion. The final product may also be dipped or soaked in a solution of sodium chloride, such as in an aqueous solution of about 0.9% sodium chloride. Desired salt content in the final product may be in the range of 0.5-1%, such as about 0.9%, of the volume of the wet product. The salts, buffers and the like agents may be provided to obtain physiological conditions.

Multivalent cations may be included to obtain non-covalent crosslinking of the nanofibrillar cellulose. One example provides a nanofibrillar cellulose product comprising nanofibrillar cellulose, especially comprising anionically modified nanofibrillar cellulose, and multivalent cations, such as multivalent metal cations, for example selected from cations of calcium, barium, magnesium, zinc, aluminum, gold, platinum and titanium, wherein the nanofibrillar cellulose is crosslinked by the multivalent cations. Especially barium and calcium may be useful in biomedical application, and especially barium may be used in labelling and can be used for detecting the injected hydrogel The amount of the multivalent cations may be in the range of 0.1-3% (w/w), for example 0.1-2% (w/w) calculated from the dry content of the hydrogel.

One example provides a method for preparing such a hydrogel, the method comprising providing pulp, disintegrating the pulp until nanofibrillar cellulose is obtained, forming the nanofibrillar cellulose into a hydrogel

The nanofibrillar cellulose may be fibrillated into a desired fibrillation degree and adjusted into desired water content, or otherwise modified, so that it forms a gel having desired properties as described herein. In one example the nanofibrillar cellulose in the hydrogel is anionically modified nanofibrillar cellulose.

The hydrogel to be used as a medical or scientific hydrogel needs to be homogenous. Therefore the method for preparing the hydrogel may include homogenizing a hydrogel comprising nanofibrillar cellulose, preferably with a homogenizing device such as ones described herein. With this preferably non-fibrillating homogenizing step it is possible to remove areas of discontinuity from the gel. A homogenous gel having better properties for the applications is obtained. The hydrogel may be further sterilized, for example by using heat and/or radiation, and/or by adding sterilizing agents, such as antimicrobials.

The present application provides use of nanofibrillar cellulose for preparing the transplantable cell composition. The nanofibrillar cellulose may be any suitable nanofibrillar cellulose disclosed herein, and the prepared transplantable cell composition may be any transplantable cell composition disclosed herein.

### Use of the Composition

The compositions comprising eukaryotic cells in a nanofibrillar cellulose hydrogel disclosed herein may be used in a variety of methods comprising delivering, transplanting, injecting, implanting and/or otherwise administering the composition to a subject, such as human or animal subject, for example a person. The subject may be a patient, especially a patient in need of therapy which involves the cells included in the composition. The methods include providing the composition comprising eukaryotic cells in a nanofibrillar cellulose hydrogel in a suitable form, such as in injectable form, implantable form or transplantable form.

The present application provides the transplantable composition for use in a therapeutic method comprising administering cells to a subject. The present application provides the transplantable composition for use in cell transplantation.

The uses may be implemented in therapeutic methods such as cell-based therapy, for example stem cell therapy. The methods may be cell transplanting methods, as disclosed herein.

One example provides a method for treating a subject in need of stem cell therapy, the method comprising
recognizing a subject in need of stem cell therapy,
providing the composition comprising eukaryotic stem cells in a nanofibrillar cellulose hydrogel disclosed herein, and
administering or delivering the composition to the subject, for example by injecting or by implanting.

One example provides a method for treating a subject in need of cell transplantation, the method comprising
recognizing a subject in need of cell transplantation,
providing the composition comprising eukaryotic cells in a nanofibrillar cellulose hydrogel disclosed herein, and
transplanting the composition to the subject, for example by injecting or by implanting.

The therapeutic methods wherein the stem cells may be used, are various and include for example tissue regeneration, cardiovascular disease treatment, brain disease treatment, such as Parkinson's and Alzheimer's disease treatment, cell deficiency therapy, such as in type I diabetes, blood disease treatments, such as providing hematopoietic stem cells for treating leukemia, sick cell anemia and other immunodeficiency problems.

### EXAMPLES

The NFC hydrogel used with stem-cell spheroids reduced undesirable agglomeration and insulated cells from injection shearing forces during cell transplantation. It was shown that human embryonic stem-cell derived spheroids (hECM) were successfully differentiated to neural networks in NFC hydrogel. hECM spheroids were successfully transplanted in NFC into the mouse inner ear where they stayed alive 3 weeks after the cell delivery.

The following experiments disclose generation of a supportive biochemical stem cell niche for a stem cell replacement therapy in the inner ear

### Introduction

Hearing loss typically results from loss of the hair cells (HC) and subsequent loss of spiral ganglion neurons (SGNs), which are the obligatory links to the brain. Following damage to the HCs, degeneration of spiral ganglion neurons (SGNs) can occur over weeks to years. In humans and most experimental animals, SGN damage follows a distal-to-central progression, with peripheral processes degenerating first. This pattern has negative implications for hearing restoration with cochlear implant (CI): with peripheral processes absent and SGN cell bodies presumed to be electrically unexcitable the electrical subjects of CI electrodes are the relatively distant central (modiolar) processes. Such “electrode-neuron gaps” decrease spatial selectivity of CI electrodes, increase deleterious CI channel interactions, and possible limit information transfer.

Implantation of an intra-scalar extracellular matrix (ECM) can provide a stem-cell niche by integrating a mechanical scaffold with the scala's squamous epithelium. ECMs can retain transplanted stem cells in place and localize cell-to-cell signalling. Their use with stem-cell derived spheroids can reduce undesirable agglomeration and insulate cells from injection shearing forces that may cause apoptosis. In the past, mammal-derived collagens were commonly used, as they provide local environment cues present in mammalian tissue. However, they are vulnerable to in vivo enzymatic degradation, making it difficult to create long-lasting niche. Nanofibrillar cellulose (NFC) hydrogels have potential for generating a stem cell niche. NFC hydrogels mimic native soft tissue ECMs in fiber size and mechanical properties. They are injectable and thus capable of delivering cells, including human embryonic stem-cell derived spheroids, to a desired subject. NFC hydrogels are easily injected, as the extruding shearing force is large enough to reduce viscosity during injection and are stable to retain their shape once the shearing force is removed. As plant-derived materials, NFC hydrogels are xeno-free. Cellulose is biocompatible due to moderate, if any, foreign responses and safe for stem-cell applications, with no known toxicity to hESCs. It is also biodurable; cellulose resorption is slow, as cells cannot synthesize cellulases required to degrade cellulose. NFC hydrogels can remain localized and act a durable carrier for in vivo drug release for example in mice. Incorporation of derived aggregates within an ECM (i.e., NFC hydrogels) promote otic neuronal differentiation, neurite outgrowth, and synaptogenesis was studied. hESC-derived spheroids as 3-D multicellular aggregates were implanted in mice inner ear with NFC hydrogels.

### Protocol

### Generation of Human Otic Neuronal Progenitors (ONPs) and SGNs from hESCs

Recapitulating the stages of human ONPs and SGNs development with a stepwise approach facilitates a controlled differentiation of hESCs toward SGN fates. A protocol was developed for deriving ONPs and SGNs from hESCs (H1, H7, and H9, WiCell, Wis., U.S.A.) through treatment with human analogs of diffusible ligands expressed in chick, Xenopus, and rodent auditory nervous systems.

### Generation of Human ONPs and SGNs from hESCs

Human ESC-derived ONPs were produced using the protocol described in Matsuoka A J, Morrissey Z D, Zhang C, Homma K, Belmadani A, Miller C A, et al. Directed differentiation of human embryonic stem cells toward placode-derived spiral ganglion-like sensory neurons. Stem Cells Transl Med. 2017 March; 6(3):923-36 and seeded into EZSPHERE™ microwells to produce spheroid aggregates, with diameter controlled by seeding density and culture time.

Shortly, the method is as follows.

1. Seed hESC derived otic neural progenitors (ONPs) in a traditional monolayer six-well tissue culture plate coated with r-Laminin-511 (iMatrix-511™, Nacalai) for two days supplemented with our previously published ONP maintenance medium

2. Single suspend ONPs and plate into micro-fabricated 3-D cell culture device (EZSPHERE™, Nacalai) at a seeding density of 2×10⁶ cells/ml.

3. Allow cells to coalesce in microwells and form aggregates. After 3 days in culture, spheroids are ready to be transferred to GrowDex.

4. Prior to transfer, GrowDex was diluted with PBS (−/−) to bring the working concentration to 1% (v/v)

5. 150 μl of either 1% native grade GrowDex or 1% anionic grade GrowDex( ) was added to a 96-well plate.

5a. GrowDex was transferred to wells using a low adhesion P200 micropipette tip

5b. GrowDex was taken up and dispensed slowly to avoid producing bubbles. If uptake was difficult, GrowDex was spun down in a mini centrifuge.

6. Using a light microscope to assist in viewing the spheroids, one to two spheroids were removed from culture using a wide-mouth P1000 micropipette tip and dispensed on top of the GrowDex and gently mixed into the hydrogel

7. 100 μl of ONP maintenance medium was then carefully placed on top of the GrowDex

8. The plate was incubated at 37° C., 5% CO₂ and one-half media changes were performed every three days by removing media from the top of the well (taking care not to disturb the gel). The appropriate amount of media and growth factors were subsequently added on top of the gel.

### Deafening and Hearing Assessments

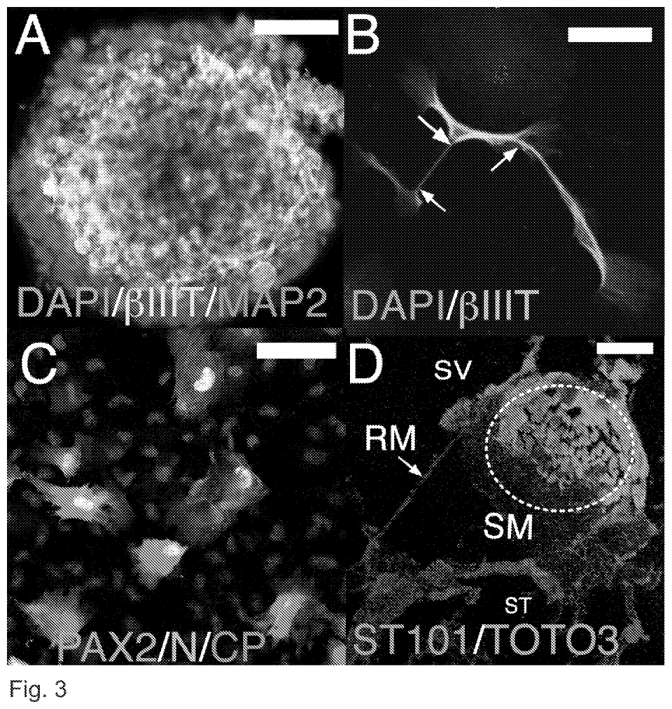

DTR mice of ages P28-30 were deafened with a single 50 ng/g i.m. injection ofdiphtheria toxin (Catalog #: D0564, Sigma-Aldrich, St. Louis, Mo.). Tone-burst auditory brainstem responses (ABR) were obtained immediately before injection and 1 week after injection to confirm efficacy. ABRs were collected within an IAC doublewall sound booth with the mouse sedated using 75 mg/kg ketamine and 8 mg/kg xylazine (i.p.). Stimulus control and averaging were performed using custom software written using the TestPoint platform. Tone bursts were generated at 250,000 sample/s with 12-bit resolution and presented with an inter-stimulus interval of 51 ms. The sinusoids were windowed with a linear 1 ms ascending ramp, a 3 ms flat plateau, and a 1 ms linear descending ramp. After impedance transformation by a unity-gain Alesis RA150 amplifier and transduction by a Beyer DT-7700 driver mounted in a custom-built speculum, the speculum is placed at the entrance of the external canal. Subdermal needle electrodes were inserted over bregma (+ input), mastoid (− input) and abdomen (indifferent ground). Evoked potentials were amplified (10,000×) by a WPI ISO-80 differential amplifier and filtered by a Frequency Devices 901P filter set (8-pole Butterworth high-pass, 3 dB cut-off frequency of 300 Hz; 8-poll Bessel low-pass, 3 dB frequency of 3 kHz). Time-domain averaging was conducted so that at least a 6 dB response-to-noise ratio is achieved or 1024 averages were collected, with waveforms stored at 250,000 sample/s and 12-bit resolution. Artifact rejection was used to omit contamination by cardiomyogenic activity (i.e., EMG), which is relatively large in the mouse. An exemplary figure of ABR on a DTR mouse and a wild-type C57/BL6J (Jackson Laboratory, Bar Harbor, Me., U.S.A.) is shown in FIG. 2.

### In Vivo Transplantation of hESC-Derived Auditory Neurons into the Inner Ear

Following five days in the 3-D culturing device, hESC-derived ONP spheroids were transferred to a 96-well plate and suspended in 1.5% GrowDex™ or 2% GrowDex-T™. Human ESC-derived ONP Spheroids were transferred using a widemouth P1000 micropipette tip and gently mixed into the GrowDex™ or GrowDex-T™ to prevent bubble formation. Once resuspended in the NFC hydrogel, the hydrogel and spheroids were transferred again to a 35-mm cell culture dish. The low walls of the 35-mm dish allow for our cell-transfer micropipette to approach the dish at an appropriately low angle.

DTR mice were implanted one week after deafening using aseptic techniques. Meloxicam (2 mg/kg) was given prior to surgery to minimize the potential complication of respiratory distress sometimes observed with concurrent anesthesia. Follow-up doses (1 mg/kg) were given once per day for three days and PRN. Anesthesia was induced using isoflurane at 3-4% and reduced to 1-2% after induction, delivered using O₂ gas at 0.3 l/min and N₂O gas at 0.25 l/min. The animal's head was secured to a custom head-holder that also delivers anaesthetic gas through a nose cone. Heat therapy was provided by a circulating water pump and insensate fluid loss replaced at 0.1 ml/10 g. Once the surgical plane of anesthesia is reached, a post-auricular incision was made and skin and muscle reflected rostrally and caudally using suture tie-downs. Visualization of the facial nerve exiting bone post-auricularly provides the landmark (immediately posterior to that point) to thin the temporal bone overlying the round window by means of a 0.5 mm diamond burr, with care taken to avoid disturbing the stapedial artery. To provide access to the scala tympani, the round window membrane was excised using a bent 33 G needle. The micropipette holder and its micromanipulators were adjusted so that the micropipette was properly contacted the margin of the round window membrane. Once completed, the entire manipulator was rotated on its vertical axis to move the pipette tip to the cell dish mounted on an elevator stand; this minimizes unwanted complications of additional adjustments and preserving hydrostatic pressure of the micropipette. The cell culture dish was thus raised to the level of the injection micropipette and spheroids were held by the micropipette by either the pressure-based (Xenoworks Digital Injector, Sutter Inc., Novato, Calif., U.S.A.) or volume-transfer based (Digital Microsyringe Pump, WPI, Inc., Sarasota, Fla., U.S.A.) instruments. Once captured, the micropipette was swung back into the transplantation position and the spheroid or organelle released into the basal turn of the cochlea. The round window defect was covered by fascia and secured with Vet Bond adhesive. Muscle and skin were closed in layers and the animal was allowed to recover with the aid of thermal therapy. Postoperatively, we administered Buprenorphine-SR-Lab for prophylactic management of pain. Recovery in a quiet and visually secluded cage generally helps reduce stimulation, aiding in stress reduction. In the case of postoperative complication (e.g., surgical wound infection) topical antibiotic was administered and pain management was carried out in consultation with veterinary staff.

### Tissue Fixation and Immunohistochemistry

After completion of the post-implantation survival period, each animal was euthanized in a CO₂ chamber followed immediately by intracardial perfusion. The chest cavity was opened, right atrium snipped, and the tip of the left ventricle pierced by a 25 G needle for perfusion. A volume of 10 ml of physiologic saline was perfused, followed by 10 ml of 4% paraformaldehyde. The cochlea was dissected from the temporal bone and placed in 5 ml 16% EDTA, continuously rotated at 4° C. for 5 days, with daily solution changes. Tissue was cryoprotected over three days with an increasing sucrose gradient (10% to 30%). Samples were embedded in Optimal Cutting Temperature Compound (F) and frozen at −80° C. Tissue was then sectioned into 10 μm slices on a Leica CM3050 S cryostat (Leica Inc., Nussloch, Germany) and mounted on gelatin-coated slides. Prior to staining, slides were stored at −80° C. Antigen retrieval was performed by steaming slides at 120° C. for 30 minutes in 0.001M EDTA (pH=9). After steaming, samples were washed three times with PBS and blocked for one hour in a solution of 10% normal goat serum and 5% bovine serum albumin in PBST. Samples were incubated overnight at 4° C. with a mouse anti-human nuclear antibody (1:100, STEM101, Takara Bio, Tokyo, Japan). The following day slides were washed with 1% normal goat serum in PBST before incubation for one hour (at room temperature and protected from light) with an Alexa Fluor 405 conjugated anti-mouse secondary antibody (1:500, Invitrogen, Waltham, Mass., U.S.A.). Slides were washed with 1% normal goat serum in PBST and then incubated for 15 minutes with a TOTO-3 iodide nuclear counterstain (1:10,000, Thermo Fisher, Waltham, Mass., U.S.A.). Samples were finally washed with PBS and then treated with Prolong Gold Antifade Mountant (Thermo Fisher, Waltham, Mass., U.S.A.). Laser scanning confocal imagery was performed with a Leica TCS SP5 microscope (Leica, Inc., Nussloch, Germany).

### Results

A step-wise neuronal differentiation protocol from undifferentiated hPSCs towards ANs is provided. Using this protocol, a hESC-derived ONP spheroid generated with 2% GrowDex™ is shown in FIG. 1. Also, a hESC-derived ONP spheroid generated with 1.5% GrowDex™ is shown in FIG. 2. Immunocytochemistry demostrates that a human-ESC derived ONP spheroid cultured with 2% GrowDex-T™ expresses AN-protein markers (MAP2 and β-III tubulin) (FIG. 3A). The hESC-derived ONP can be further differentiated into a more mature neuron. FIG. 3B shows hESC-derived ONP cultured in neuronal differention medium for additional 14 days extends neurites among each other; suggesting that they have established a neuronal network. To indentify a hESC-derived ONP spheroid in vivo, hESC-derived ONPs that express GFP in the nucleus and RFP in cytoplasm (FIG. 3C) were generated using a pLOC lentiviral vector containing the Evrogen TurboRFP ORF and the TurboGFP-2A-Blast (GE Healthcare, Chicago, Ill., U.S.A.). Human ESC-derived ONP spheroids transplanted with 1.5% GrowDex™ in the DTR mouse inner ear can be identified with STEM101 (ST101), anti-human nuclear antibody. Note that STEM101 positive cells are found in the scala media (SM), one of the three chambers in the inner ear, three weeks after a transplantation surgery with four hESCderived ONP spheroids.

1. A method for preparing transplantable cell preparation, the method comprising
culturing eukaryotic cells at conditions allowing the cells to form cell aggregates, which are cell spheroids,
providing the cell aggregates in a nanofibrillar cellulose hydrogel and/or combining the cell aggregates with a nanofibrillar cellulose hydrogel to obtain a transplantable cell composition comprising eukaryotic cells in a nanofibrillar cellulose hydrogel matrix, the composition containing no or only traces of other type of cell-derived material and/or wherein the comoposition is animal origin free, xeno-free and/or feeder-free.

2. The method of claim 1, comprising
removing cell culture medium before providing the cell aggregates in the nanofibrillar cellulose hydrogel and/or combining the cell aggregates with a nanofibrillar cellulose hydrogel by filtering, centrifuging and/or washing the cell aggregates with a medium containing no other type of cell-derived material, and/or which is animal-origin free, xeno-free and/or feeder-free.

3. The method of claim 1, comprising culturing the eukaryotic cells in cell culture medium containing no other type of cell-derived material, and/or which is animal-origin free, xeno-free and/or feeder-free.

4. The method of claim 1, comprising culturing the eukaryotic cells in nanofibrillar cellulose hydrogel having a concentration in the range of 0.8-3% (w/w).

5. The method of claim 1, wherein the nanofibrillar cellulose has an average diameter of a fibril in the range of 1-200 nm.

6. The method of claim 1, wherein the nanofibrillar cellulose, when dispersed in water, provides a zero shear viscosity in the range of 1000-100000 Pa·s and a yield stress in the range of 1-50 Pa, determined by rotational rheometer at a consistency of 0.5% (w/w) by weight in aqueous medium at 22° C.±1° C.

7. The method of claim 1, wherein the nanofibrillar cellulose, when dispersed in water, provides a storage modulus in the range of 350-5000 Pa and yield stress in the range of 25-300 Pa determined by stress controlled rotational rheometer with gradually increasing shear stress in a range of 0.001-100 Pa at a frequency 10 rad/s, strain 2%, at 25° C.

8. The method of claim 1, wherein the cells are stem cells.

9. The method of claim 1, wherein the cell spheroids have an average diameter in the range of 80-700 μm.

10. A transplantable cell composition comprising eukaryotic cell spheroids in a nanofibrillar cellulose hydrogel matrix, the composition containing no or only traces of other type of cell-derived material and/or wherein the composition is animal origin free, xeno-free and/or feeder-free.

11. The transplantable cell composition of claim 10, wherein the cells are stem cells.

12 (canceled)

13. The transplantable cell composition of claim 11, wherein the cell spheroids have an average diameter in the range of 80-700 μm.

14 (canceled)

15. The transplantable cell composition of claim 11, wherein the stem cells are selected from mesenchymal stem cells, multipotent adult progenitor cells, induced pluripotent stem cells, and hematopoietic stem cells

16. The transplantable cell composition of claim 11, wherein the stem cells are human stem cell selected from non-embryonic cells and embryonic cells, which have been derived without destroying the embryo.

17. The transplantable cell composition of claim 10, wherein the cells are differentiated cells.

18. The transplantable cell composition of claim 10, wherein the concentration of the nanofibrillar cellulose in the nanofibrillar cellulose hydrogel matrix is in the range of 1-3% (w/w).

19. The transplantable cell composition of claim 10, wherein the nanofibrillar cellulose comprises chemically unmodified nanofibrillar cellulose.

20. The transplantable cell composition of claim 10, wherein the nanofibrillar cellulose comprises chemically modified nanofibrillar cellulose selected from anionically modified nanofibrillar cellulose, oxidized nanofibrillar cellulose and TEMPO oxidized nanofibrillar cellulose.

21. The transplantable cell composition of claim 10, wherein the nanofibrillar cellulose has an average diameter of a fibril in the range of 1-200 nm.

22. The transplantable cell composition of claim 10, wherein the nanofibrillar cellulose, when dispersed in water, provides a zero shear viscosity in the range of 1000-100000 Pa·s and a yield stress in the range of 1-50 Pa, determined by rotational rheometer at a consistency of 0.5% (w/w) by weight in aqueous medium at 22° C.±1° C.

23. The transplantable cell composition of claim 10, wherein the nanofibrillar cellulose, when dispersed in water, provides a storage modulus in the range of 350-5000 Pa and yield stress in the range of 25-300 Pa, determined by stress controlled rotational rheometer with gradually increasing shear stress in a range of 0.001-100 Pa at a frequency 10 rad/s, strain 2%, at 25° C.

24. (canceled)

25. The transplantable cell composition of claim 10 in a form of an implant.

26. The transplantable cell composition of claim 10 wherein the composition is an injectable composition or an implantable composition

27. An implant comprising transplantable cell composition comprising eukaryotic cells in a nanofibrillar cellulose hydrogel matrix of claim 10

28. (canceled)

29. (canceled)

30. A method for treating a subject in need of cell transplantation, the method comprising
recognizing a subject in need of cell transplantation,
providing the composition comprising eukaryotic cells in a nanofibrillar cellulose hydrogel of claim 10, and
transplanting the composition to the subject.

31. The method of claim 1, wherein the concentration of the nanofibrillar cellulose in the nanofibrillar cellulose hydrogel matrix is in the range of 1-3% (w/w).
